(19) United States
(12) Patent Application Publication
(10) Pub. No.: US 2004/0249218 A1
(43) Pub. Date: Dec. 9, 2004

(54) Method for producing 6-methylheptane-2-one and the use thereof

(76) Inventors: Klaus-Diether Wiese, Haltern am See (DE); Guido Protzmann, Zwingenberg (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/490,451

(22) PCT Filed: Sep. 27, 2002
(86) PCT No.: PCT/EP02/10873

(30) Foreign Application Priority Data
Oct. 6, 2001 (DE) 101 49 349.5

(51) Int. Cl. C07C 45/49

(52) U.S. Cl. 568/387; 568/388

(57) ABSTRACT

Process for preparing 6-methylheptan-2-one, which comprises

a) hydroformylation of isobutene to form 3-methylbutanal,

b) base-catalyzed aldol condensation of the 3-methylbutanal with acetone to form 6-methylhept-3-en-2-one, with the molar ratio of 3-methylbutanal to the base used being more than 1:0.3 and

c) hydrogenation of the 6-methylhept-3-en-2-one to give 6-methylheptan-2-one.

The use of the 6-methylheptan-2-one prepared in this way for the preparation of isophytol, tetrahydrolinalool or dihydrogeraniol.

The present invention relates to a three-stage process for preparing 6-methylheptan-2-one from isobutene and the use of the product prepared in this way.

6-Methylheptanone is an intermediate in the preparation of isophytol, a building block for the synthesis of vitamin E. Furthermore, it is a starting material for the synthesis of tetrahydrolinalool, dihydrogeraniol and further flavors.

Various synthetic routes for preparing 6-methylheptan-2-one are known from the literature.

Reaction of 3-methylbutyl halides with acetoacetic esters in the presence of bases forms an intermediate whose hydrolysis and decarboxylation gives 6-methylheptan-2-one (Wagner et al in Synthetic Organic Chemistry, p. 327, John Wiley & Sons, Inc.). This synthesis has a number of disadvantages: due to the price of acetoacetic esters, in particular, the raw material costs are high. At least equimolar amounts of base are to be used. A halide is formed as by-product and has to be disposed of.

The title compound can also be obtained by hydrogenation of 6-methyl-5-hepten-2-one or 6-methyl-3,5-heptadien-2-one over nickel or other catalysts (Izv. Akad. Nauk SSSR, Ser. Khim (5) (1972) 1052). Since both these starting materials are expensive, the target product cannot be prepared economically in this way.

EP 0 816 321 A discloses a two-stage process for preparing 6-methylheptan-2-one. In the first stage, 3-methylbutanal is aldol-condensed with acetone. In the second stage, the crude product is hydrogenated to the target product. The aldol condensation is carried out batchwise in an autoclave at a pressure of 1.9 bar and a temperature of 72° C. Acetone is initially placed in the autoclave and 3-methylbutanal and 2% strength aqueous sodium hydroxide solution are added dropwise over a period of 175 minutes. After cooling to room temperature, the organic phase is separated off. This is hydrogenated over 5% Pd/activated carbon for 7 hours at 120° C. and a pressure of 5-9 bar. After the catalyst has been filtered off, the crude hydrogenation product is worked up by distillation. The yield of target product over both stages is 62% based on 3-methylbutanal. This process has the disadvantages that both stages have to be carried out batchwise, with a relatively long cycle time, which in turn results in low space-time yields.

EP 0 765 853 describes a further two-stage process for preparing 2-methylheptan-2-one. In the first stage, 3-methylbutanal is reacted with acetone to form 4-hydroxy-6-methylheptan-2-one and a small yield of 6-methyl-3-hepten-2-one. To increase the selectivity, this is carried out by reaction of the aldehyde with acetone in a molar ratio of from 1:3 to 1:10 using a base in a molar ratio to the aldehyde of from 0.1 to 20%.

The small addition of base is intended to increase the selectivity, i.e. avoid self-condensation of the aldehyde or the acetone. However, a disadvantage of carrying out the reaction in this way in an industrial process is the very low space-time yield.

In the second stage, this mixture is hydrogenated with simultaneous elimination of water. In the first stage, aqueous alkali metal hydroxides or alkaline earth metal hydroxides are used as catalyst. After reaction is complete, the reaction mixture is neutralized with acetic acid, acetate which precipitates is filtered off and the two intermediates are isolated by distillation. The distillate is hydrogenated in the presence of an acid (p-toluene sulfonic acid) at 100° C. and a pressure of 8 bar over a catalyst comprising 5% Pd/activated carbon. The catalyst is filtered off from the crude hydrogenation product, the organic phase is separated off and the target product is separated therefrom by distillation. The yield of 6-methylheptan-2-one over both stages is 65% based on 3-methylbutanal. This process has some disadvantages: the base used in the first stage is neutralized with acetic acid. As a result, the process is encumbered by additional raw material costs. The acetates formed have to be disposed of, which incurs further costs.

From an economic point of view, the known processes do not yet meet all demands made of a process carried out on an industrial scale, either because the starting materials are not available in a sufficient quantity and/or at low cost or because the conversion of the starting materials into 6-methylheptan-2-one is associated with a process which is too complicated.

It is therefore an object of the invention to develop a more economical process for the industrial preparation of 6-methylheptan-2-one on the basis of inexpensive and readily available materials.

It has been found that 6-methylheptan-2-one can be obtained in large quantities from isobutene by hydroformylation to form valeraldehyde, aldol condensation of the latter with acetone and finally hydrogenation of the aldolization product.

The present invention accordingly provides a process for preparing 6-methylheptan-2-one, which comprises

a) hydroformylation of isobutene to form 3-methylbutanal,

b) base-catalyzed aldol condensation of the 3-methylbutanal with acetone to form 6-methylhept-3-en-2-one, with the molar ratio of 3-methylbutanal to the base used being more than 1:0.3 and

c) hydrogenation of the 6-methylhept-3-en-2-one to give 6-methylheptan-2-one.

The 6-methylheptan-2-one prepared according to the invention can be used for the preparation of isophytol, tetrahydrolinalool or dihydrogeraniol.

The isobutene used as starting material for the preparation of 6-methylheptan-2-one by the process of the invention can come from many sources. Isobutene can be used as a pure substance or as an isobutene-containing mixture, e.g. with further C₄ hydrocarbons. Industrial mixtures in which isobutene is present are the C₄ fraction from an FCC, the C₄ fraction from a steam cracker, raffinate I obtained from the C₄ fraction from a steam cracker by butadiene extraction or a hydrogenated C₄ fraction from a steam cracker, in which the major part of the butadiene has been selectively hydrogenated to linear butenes. Further isobutene-containing streams include mixtures which have been obtained by dehydrogenation of isobutane-containing hydrocarbon streams.

Furthermore, isobutene-rich streams are also produced by skeletal isomerization of C₄ streams comprising linear butenes.

Isolation of isobutene from a C₄ fraction is in principle carried out by two work-up processes. The first step which is common to both work-up variants is the removal of the major part of the butadiene. If butadiene can be readily marketed or is consumed in-house, it is separated off by extraction or extractive distillation. Otherwise, it is selectively hydrogenated to linear butenes so as to leave a residual concentration of about 2000 ppm. In both cases, the product is a hydrocarbon mixture (raffinate I or hydrogenated cracker C₄) comprising the saturated hydrocarbons n-butane and isobutane together with the olefins isobutene, 1-butene and 2-butenes.

Isobutene is separated off from this hydrocarbon mixture by reaction with methanol to form methyl tert-butyl ether (MTBE). The redissociation of MTBE gives a mixture of methanol and isobutene which can easily be separated into the two components. Isobutene can be isolated analogously by reaction with water to form the intermediate tert-butanol and redissociation of the latter.

As an alternative, a virtually butadiene-free C₄ fraction (C₄ stream from an FCC, raffinate I or hydrogenated cracker C₄) can be hydroisomerized in a reactive column. This gives a product at the top of the column which comprises isobutane and isobutene.

Hydroformylation (Step a)

The hydroformylation of isobutene by means of synthesis gas to give 3-methylbutanal is known. Cobalt or rhodium catalysts can be used in this reaction. In cobalt-catalyzed hydroformylation (DE 39 02 892 A1), the yield is up to 74%. In addition, 2,2-dimethylpropanal and isobutane are formed. Better yields are achieved in hydroformylation using rhodium catalysts in the presence of organic phosphite ligands. The hydroformylation of isobutene to 3-methylbutanal using a catalyst system comprising rhodium and a bisphosphite is disclosed in, for example, U.S. Pat. No. 4,668,651, U.S. Pat. No. 4,769,498 and WO 85-03702. U.S. Pat. No. 4,467,116 describes, inter alia, the terminal hydroformylation of a-olefins which are dialkylated in the 2 position. This is carried out using catalyst systems comprising rhodium and a triarylphosphine in which at least one aryl radical bears a bulky substituent in the ortho position.

Step a) (hydroformylation) of the process of the invention can be carried out using a catalyst system comprising rhodium and a phosphite having the structure I.

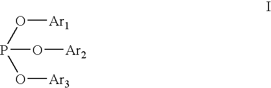

Here, Ar₁, Ar₂ and Ar₃ are aromatic radicals which can be identical or different and be substituted or unsubstituted. Suitable aromatic radicals are, for example, phenyl, naphthyl, phenanthryl or anthracenyl. At least one of the aromatic radicals bears a group R₁ in the ortho position relative to the phosphite oxygen or a further substituent X₁ in the m or p position. R₁ can in turn be aliphatic, cycloaliphatic, aromatic or heterocyclic. Purely aliphatic radicals have the structure II.

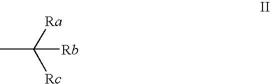

R, Rb and Rc can be identical or different and are hydrocarbon radicals having from 1 to 6 carbon atoms. R₁ is preferably a phenyl or tert-butyl group.

X₁ is a hydrocarbon or ether radical having from 1 to 6 carbon atoms.

The hydroformylation of isobutene or a hydrocarbon mixture comprising isobutene as the only unsaturated compound in step a) is preferably carried out using the above-described catalyst system comprising rhodium and a triaryl phosphite in a homogeneous reaction (one liquid phase). The reaction is carried out in a temperature range from 60° C. to 180° C., preferably in the range from 90° C. to 150° C. The reaction pressure is from bar to 200 bar, preferably from 20 bar to 100 bar. As hydroformylating agent, use is made of a mixture of carbon monoxide and hydrogen in a molar ratio of from 1/10 to 10/1. The rhodium concentration is from 5 to 500 ppm by weight, preferably from 10 to 200 ppm by weight. From 1 to 50 mol of triaryl phosphite, preferably from 5 to 30 mol, are used per mol of rhodium. The reaction can be carried out batchwise, but preference is given to a continuous procedure.

The crude reaction product is advantageously separated into unreacted isobutene, 3-methylbutanal, high boilers in which the catalyst is present and by-products by distillation. Unreacted isobutene and the catalyst are returned to the hydroformylation reactor.

Aldol Condensation (Step b)

The aldol condensation of 3-methylbutanal with acetone to form 6-methylhept-3-en-2-one is preferably carried out as a two-phase reaction. The reaction in step b) can be carried out continuously or batchwise, in a tube reactor, flow tube or in a stirred vessel.

The aldol condensation is base-catalyzed, and preferred bases are inorganic, aqueous systems having a base concentration of from 0.1 to 15% by weight. Useful bases are alkali metal hydroxides such as NaOH, KOH, K₂O, Na₂O or NaHCO₃, Na₂CO₃, K₂CO₃, acetates, formates or triethylamine.

Not only the desired product 6-methylhept-3-en-2-one but also the by-products 4-methyl-2-penten-2-one (4-MP), 3-methyl-2-isopropyl-2-butenal (3-MiPB), 5-methyl-2-isopropyl-2-hexenal (5-MiPH), 4-hydroxy-6-methylheptan-2-one (6-HMH) are formed in the aldol condensation. The compounds are, for example, also present as enol tautomers, and for the purposes of the present invention the desired product encompasses all tautomeric forms of 6-methylhept-3-en-2-one.

The mass ratio of 3-methylbutanal to the base used is above 0.3, preferably in the range from 1:1 to 1:2, very particularly preferably in the range from 1:1 to 1:5.

In a particular process variant, step b) is carried out by dispersion of an organic phase comprising methylbutanal in a continuous phase comprising the catalyst.

The reaction can, as described in the patent application DE 101 06 186.2 (method of carrying out multiphase reactions, in particular the condensation of aldehydes with ketones), in a tube reactor, with the catalyst being present in the continuous phase and the starting material being present in an organic, disperse phase and the loading factor B of the reactor being equal to or greater than 0.8 and the mass ratio of continuous phase to disperse phase being greater than 2. (The loading factor B is defined as follows: B=PD/PS. PD[Pa/m] is a length-based pressure drop over the reactor under operating conditions and PS [Pa/m] is a mathematic parameter having the dimension of length-based pressure, defined as the ratio of mass flow M [kg/s] of all components under operating conditions multiplied by g=9.81 [m/s^{2]}, i.e. PS−(M/V)+*g). As catalyst phases, preference is given in all variants of the process to aqueous solutions of hydroxides, hydrogencarbonates, carbonates or carboxylates in the form of their alkali metal or alkaline earth metal compounds, in particular sodium hydroxide and potassium hydroxide. The concentration of the catalyst in the catalyst solution is in the range from 0.1 to 15% by mass, in particular from 0.1 to 5% by mass. Further details of the reactor and its mode of operation are given in the disclosure of DE 101 06 186.

Advantageously, 3-methylbutanal, acetone and optionally a solvent are fed into the catalyst phase upstream of the respective reactor.

The molar ratio of 3-methylbutanal to acetone is from 5/1 to 1/10, preferably from 1/1 to 1/5. The reaction is carried out in a temperature range from 40° C. to 150° C., preferably in the range from 50° C. to 120° C. The reaction time is from 0.1 to 20 minutes, preferably from 0.2 to 5 minutes.

If appropriate, the catalyst phase is separated off from the crude reaction product and is returned to the reactor. Unreacted starting materials, some product, water and any solvent are preferably distilled off prior to the phase separation. After condensation, the distillate separates into an aqueous phase and an organic phase which can be returned to the reactor. The aqueous phase is preferably, after starting materials, in particular acetone, have been separated off by distillation, partly discarded for discharging the water or reaction and partly returned to the process after optional use as washing liquid.

The product phase which has been separated off from the catalyst can, if appropriate after washing with water, be worked up by distillation to give pure 2-methylhept-3-en-2-one. A further possibility is to use the crude product which has been separated off from the catalyst in the next stage. This procedure makes it possible to prepare the desired α,β-unsaturated ketone in a selectivity of 95% based on 3-methylbutanal.

In all variants of step b), it is possible to use a solvent. The use of a solvent frequently results in an increase in the selectivity of the aldol condensation, control of the loss of water from the catalyst solution and simplification of the separation of water from the aldol condensate.

Preference is given to using a solvent in which 3-methylbutanal, acetone and 6-methylhept-3-enone are soluble and the base or the continuous phase are not soluble.

Such a solvent should have the following properties: it dissolves products and starting materials and is itself very sparingly soluble in the catalyst phase. It is inert in the aldol condensation and optionally in the hydrogenation. It can be separated by distillation from the target products 6-methyl-hept-3-en-2-one and/or 6-methylheptan-2-one. Suitable solvents are, for example, ethers or hydrocarbons such as toluene or cyclohexane. In particular, preference is given to solvents which form a minimum heteroazeotrope with water, so that the water can be separated off from the aldol condensate particularly simply. For this reason, cyclohexane or toluene is preferred as solvent.

Hydrogenation (Step c)

The 6-methylhept-3-en-2-one obtained by crossed aldol condensation is, either in pure form or as a mixture which may further comprise acetone, 3-methylbutanal, water, solvent and high boilers, selectively hydrogenated to give 6-methylheptan-2-one. This is preferably carried out over fixed-bed catalysts and/or acid catalysts. Acid catalysts frequently comprise acidic support material or support material impregnated with acidic substances.

The hydrogenation is carried out using catalysts which may comprise palladium, platinum, rhodium and/or nickel as hydrogenation-active components. These metals can be used in pure form, as compounds with oxygen or as alloys. Preferred catalysts are those in which the hydrogenation-active metal has been applied to a support. Suitable support materials are aluminum oxide, magnesium oxide, silicon oxide, titanium dioxide and their mixed oxides and also activated carbon. Among these catalysts, particularly preferred catalysts are palladium on activated carbon and palladium on aluminum oxide.

In the case of catalysts comprising palladium and a support, the palladium content is from 0.1 to 5% by mass, preferably from 0.2 to 1% by mass. The hydrogenation can be carried out continuously or batchwise and either in the gas phase or in the liquid phase. Hydrogenation in the liquid phase is preferred because the gas-phase process consumes more energy as a result of the necessity of circulating large volumes of gas. Various process variants can be chosen for the continuous liquid-phase hydrogenation. It can be carried out adiabatically or virtually isothermally, i.e. with a temperature rise of less than 10° C., in one or more stages. In the latter case, the reactors can both be operated adiabatically or virtually isothermally or one can be operated adiabatically and the other virtually isothermally. Furthermore, it is possible to carry out the selective hydrogenation in a single pass or with product recirculation. The hydrogenation is carried out in a mixed liquid/gas phase or in the liquid phase in cocurrent in three-phase reactors, with the hydrogen being finely dispersed in a manner known per se in the liquid to be hydrogenated. In the interests of uniform liquid distribution, improved removal of the heat of reaction and a high space-time yield combined with high selectivity, the reactors are preferably operated at high liquid throughputs of from 15 to 300 m³, in particular from 25 to 150 m³, per m² of cross section of the empty reactor and hour. One hydrogenation process for preparing 6-methylheptan-2-one is, for example, liquid-phase hydrogenation in two or more reactors which are all operated with product recirculation, as described in U.S. Pat. No. 5,831,135.

The selective hydrogenation of 6-methylhept-3-en-2-one to form 6-methylheptan-2-one in the process of the invention is carried out in the temperature range from 0 to 200° C., in particular from 40 to 150° C. The reaction pressure is from 1 to 200 bar, preferably from 1 to 30 bar, in particular from 1 to 15 bar.

Selective hydrogenation offers the advantage that the target product is obtained in a yield of above 99% at virtually 100% conversion. Any saturated carbonyl compounds such as 3-methylbutanal or acetone present in the starting material are not hydrogenated to any significant degree.

If pure 6-methylhept-3-en-2-one is hydrogenated, i.e. an appropriate purification step (e.g. distillation) is carried out prior to the hydrogenation, the target product is obtained in a sufficiently good quality for further purification to be superfluous.

On the other hand, if a crude aldol condensation mixture is fed to the hydrogenation stage, the crude hydrogenation product has to be worked up by distillation. Apart from the target product, acetone and 3-methylbutanal are also separated off. The two latter substances are returned to the aldol condensation stage.

The 6-methylheptan-2-one prepared by the process of the invention is an intermediate for the preparation of isophytol, a building block for the synthesis of vitamin E. This compound is also used for the preparation of tetrahydrolinalool, dihydrogeraniol and further flavors.

The following examples illustrate the invention but do not restrict its scope which is defined by the claims.

### EXAMPLE 1

### Hydroformylation

The experiment was carried out in an experimental plant comprising a bubble column reactor, a thin film evaporator and a distillation apparatus. The isobutene was introduced into the bubble column from below together with an excess of synthesis gas and a solution of the catalyst in a high-boiling solvent. At the top of the reactor, unreacted synthesis gas was separated off. The liquid components (residual olefin, aldehydes, by-products, high-boiling solvent, catalyst) were passed to the thin film evaporator which was operated under reduced pressure so that the aldehyde formed together with the unreacted olefins was separated from the higher-boiling components in which the catalyst was dissolved. As high-boiling solvent, use was made of dioctyl phthalate which was present in the reactor in a proportion of 20% by weight because no high boiler from the process was present when the experiment was started and would be formed only a small amount during the time of the experiment. The rhodium concentration in the reactor was 30 ppm of rhodium, and tris(2,4-di-tert-butylphenyl) phosphite was added as ligand. The P/Rh ratio was 20/1. The bubble column was maintained at a constant 115° C. by cooling externally via a double wall. The operating pressure was 50 bar of synthesis gas.

Under the above-described reaction conditions, an olefin feed rate of 2 kg/h of isobutene was set, and the bubble column had a volume of 2.1 liters. Balancing of the mass flows gave the following product distribution for isobutene and downstream products:

| | |
|---|---|
| Isobutene | 8.2 |
| Pivalaldehyde | 0.1 |
| 3-methylbutanal | 90.8 |
| 3-methylbutanol | 0.3 |
| High boilers | 0.6 |

The conversion of isobutene was 92% at a selectivity to 3-methylbutanal of 99% based on isobutene.

### EXAMPLE 2

### Aldol Condensation

The aldolization was carried out in an experimental apparatus as shown schematically in FIG. 1. In this apparatus, the continuous catalyst phase 2 was circulated by means of a pump 1. Aldehyde and ketone were fed in together through line 3 or separately through lines 3 and 4 and mixed into the catalyst. In this example, the starting materials were mixed exclusively via line 3. The multiphase mixture 5 was pumped through the tube reactor 6 which had a length of 3 m and a diameter of 17.3 mm and was provided with static mixing elements having a hydraulic diameter of 2 mm. The resulting mixture 7, comprising the reaction product, unreacted starting material and the catalyst, could be freed of volatile constituents in the gas separator 8 by discharging into line 9. In this example, this line was closed.

The liquid stream 10 obtained after degassing 8 is fed into a phase separation vessel 11. Here, the aqueous catalyst phase 2 is separated off and fed back into the circuit. The organic phase which flows out over a weir and comprises the reaction product is taken off from line 12.

The heat of reaction can be removed via heat exchangers 13, 14 and 15 located outside the reactor.

Water and acetone were used as solvent for the catalyst. The first table accompanying the example reports firstly the catalyst composition in percent by mass and then the amount of starting material and its composition in percent by mass according to analysis by gas chromatography.

In the lower part of the second table, the product composition is listed, likewise in percent by mass according to analysis by gas chromatography.

In the upper part of the second table, the space-time yield (STY), the conversion (C) of the aldehydes, the selectivity (S) to the desired aldol condensation products and the loading factor (B) are reported. In the case of the catalyst composition described, it should be noted that the values given in the examples are initial values. The proportion of NaOH was diluted slightly by the water or reaction from the aldol condensation. Furthermore, the Cannizzaro reaction which proceeds in parallel to the aldol condensation leads to neutralization of the alkaline catalyst. Both effects are, however, so small over the time observed that they are not significant for the description of the experiments and the experimental results.

This example describes the process of the invention for the aldol condensation of acetone (Ac) and 3-methylbutanal (3-MBA) in cyclohexane (CH) to form 6-methyl-3-hepten-2-one (6-MH). The formation of the by-products 4-methyl-3-penten-2-one (4-MP), 3-methyl-2-isopropyl-2-butenal (3-MiPB), 5-methyl-2-isopropyl-2-hexanal (5-MiPH) and 4-hydroxy-6-methylheptan-2-one (6-HMH) and the other high boilers (HB) is reported in percent by weight in the following table.

The reactants were passed through the reactor at a catalyst throughput of 400 kg/h at a temperature of 80° C. and the autogenous pressure of the reactants.

| | |
|---|---|
| Catalyst [kg] | 4.5 |
| c NaOH [%] | 6.7 |
| Water [%] | 89.2 |
| Acetone | 4.1 |
| Starting material [l/h] | 5.24 |
| Ac [% by weight] | 42.36 |
| 3-MBA [% by weight] | 33.34 |
| CH [% by weight] | 24.30 |

The following result was achieved: (analysis without cyclohexane).

| | |
|---|---|
| STY [t/m³/h] | 3.2 |
| C | 0.86 |
| S | 0.95 |
| B | 15.34 |
| Ac | 33.27 |
| 3-MBA | 5.26 |
| 6-MH | 58.37 |
| 4-MP | 0.66 |
| 3-MiPB | 0.42 |
| 5-MiPH | 0.3 |
| 6-HMH | 0.5 |
| HB | 1.2 |

It can clearly be seen that 6-methyl-3-methylhepten-2-one can be prepared in a higher selectivity at greater space-time yields by means of the process of the invention.

### EXAMPLE 3

### Hydrogenation

The hydrogenation of 6-methyl-3-hepten-2-one (6-MH) to 6-methylheptan-2-one (6-MHa) in this example was carried out in a differential circulation reactor under isothermal and isobaric conditions. 70 g of a Pd/Al₂O₃ catalyst were used as catalyst. The fixed bed had a diameter of 4 mm. The catalyst used was reduced beforehand at 80° C. and a hydrogen pressure of bar for 18 hours. The volume flow of the reaction mixture in the circuit was 45 l/h. This corresponds to a cross-sectional loading of 35 m³/m²/h.

The following table reports the product analysis of the reaction mixture in percent by weight after a reaction time of 5 hours. Apart from the starting material and the product, analyses were carried out for 6-methylheptan-2-ol (6-MHO) and high boilers (HB).

| | |
|---|---|
| 6-MH | 0.49 |
| 6-MHa | 98.51 |
| 6-MHO | 0.15 |
| HB | 0.85 |

The conversion of 6-methyl-3-hepten-2-one is 99.5% at a selectivity to 6-methylheptan-2-one of 99%.

1: A process for preparing 6-methylheptan-2-one, which comprises
a) hydroformylation of isobutene to form 3-methylbutanal,
b) base-catalyzed aldol condensation of the 3-methylbutanal with acetone to form 6-methylhept-3-en-2-one, with the molar ratio of 3-methylbutanal to the base used being more than 1:0.3 and
c) hydrogenation of the 6-methylhept-3-en-2-one to give 6-methylheptan-2-one.

2: The process as claimed in claim 1, wherein the hydroformylation in step a) is carried out in the presence of a cobalt or rhodium catalyst.

3: The process as claimed in claim 1, wherein the hydroformylation in step a) is carried out in the presence of a rhodium catalyst and an organic phosphite ligand.

4: The process as claimed in claim 1, wherein the base used in step b) is an aqueous inorganic base in a concentration of from 0.1 to 15% by weight.

5: The process as claimed in claim 4, wherein the base used is sodium hydroxide.

6: The process as claimed in claim 1, wherein step b) is carried out in a tube reactor.

7: The process as claimed in claim 6, wherein the loading factor of the tube reactor is greater than 0.8.

8: The process as claimed in claim 6, wherein step
b) is carried out by dispersion of an organic phase comprising methylbutanal in a continuous phase comprising the catalyst.

9: The process as claimed in claim 8, wherein the mass ratio of the continuous phase to the disperse, organic phase is greater than 2.

10: The process as claimed in claim 1, wherein step b) is carried out using a solvent in which 3-methylbutanal, acetone and 6-methylhept-3-en-2-one are soluble and the base or the continuous phase is insoluble.

11: The process as claimed in claim 10, wherein the solvent forms a minimum azeotrope with water.

12: The process as claimed in claim 1, wherein the hydrogenation is carried out over a catalyst which is present in a fixed bed.

13: The process as claimed in claim 12, wherein the hydrogenation is carried out over an acid catalyst.

14: The process as claimed in claim 12, wherein the hydrogenation is carried out over a palladium catalyst.

15: A method for preparing isophytol, tetrahydrolinalool, or dihydrogeraniol, which comprises:
reacting the 6-methylheptan-2-one as prepared in claim 1 for the preparation of isophytol, tetrahydrolinalool or dihydrogeraniol.
